(19) United States
(12) Patent Application Publication
(10) Pub. No.: US 2007/0016210 A1
(43) Pub. Date: Jan. 18, 2007

(54) Device and method for use to create multiple bone grafts for use in fusion

(76) Inventor: Frank H. Boehm JR., Utica, NY (US)

Correspondence Address: FRANK H. BOEHM, JR., MD, 2408 GENESEE STREET, UTICA, NY, 13502, US

(21) Appl. No.: 11/485,688

(22) Filed: Jul. 13, 2006

Related U.S. Application Data
(60) Provisional application No. 60/700,113, filed on Jul. 13, 2005.

(51) Int. Cl. A61B 17/00

(52) U.S. Cl. 606/79

(57) ABSTRACT

The inventors provide a device and method for use to obtain multiple bone grafts using a technically easy, one-step method is provided. The primary site(s) for harvesting these bone grafts, as described herein, is the spinous process. However, any donor site to provide bone graft for any recipient fusion site is conceivable. The principal use disclosed herein would be in fusion procedures of the spine. The device includes a single or double action instrument, which can be manually deployed, or deployed with the assistance of any source of power. The surgeon would deploy the instrument by interfacing the trailing end of the device. the leading end of the device is comprised of a dual arms; one contains a chamber which contains multiple bone cutting edges, and the other arm provides a flattened surface against which the bone cutting edges can be brought. After the instrument is seated at the base of the spinous process, or the selected donor site, it is then deployed as the result of the engagement of the multiple bone cutting edges. The resultant effect is the division of the donor site such as the spinous process into multiple sections. These can then be removed and packed into the selected site of fusion. The inventors contemplate both the use of the device to harvest bone from other locations, as well as the use of the grafts obtained for fusions elsewhere.

This application is based on and claims priority to U.S. provisional application No. 60/700,113 filed with the United States Patent and Trademark Office on Jul. 13, 2005, and fully incorporated herein by reference.

References

| | | |
|---|---|---|
| 5,026,375 | Linovitz, etal. | Jun. 25, 1991 |
| 5,273,519 | Koros, etal. | Dec. 28, 1993 |
| 5,451,227 | Michelson | Sept. 19, 1995 |
| 5,569,258 | Gambale | Oct. 29, 1996 |
| 5,925,050 | Howard, III | Jul. 20, 1999 |
| 6,126,674 | Janzen | Oct. 3, 2000 |
| 6,142,997 | Michelson | Nov. 7, 2000 |
| 6,200,320 | Michelson | Mar. 13, 2001 |
| 6,685,710 | Agbodoe | Feb. 3, 2004 |
| 6,695,849 | Michelson | Feb. 24, 2004 |
| 6,723,103 | Edwards | Apr. 20, 2004 |
| 6,986,772 | Michelson | Jan. 17, 2006 |
| 7,011,663 | Michelson | Mar. 14, 2006 |

### BACKGROUND OF INVENTION

1. Field of Invention

The invention relates to the general field of bone fusion, and specifically to a device which resects a segment of donor bone and simultaneously divides that segment into multiple bone grafts.

2. History of Related Art

Surgical intervention for spinal disorders has been performed for more than a century. In the early part of the 20^{th} century, the concept of performing a procedure for correcting excessive movement of two or more vertebrae of the spine, referred to as the fusion, was first introduced, and has become one of the most widely performed surgical procedures in the United States and around the world. The latest estimates suggest that over 300,000 such procedures were performed in the United States alone in 2004, and that perhaps as many as 750,000 spinal fusions were performed worldwide. Despite the introduction of technologies such as prosthetic disc replacement devices, as well as posterior dynamic stabilization, it is very clear that spinal fusion continue to play a significant role in treatment of disorders of the spine for the foreseeable future.

Bone fusion is a surgical procedure by which two or more bones or fragments are united by a bony or osseous bridge, thus creating a single functional unit. Fusion may be performed at any site, including joints as well as sites of bone fractures. For the purposes of this application, fusion shall heretofore refer to spinal fusion, but it is noted that the same principals can apply to fusion at any other site.

Spinal fusion is the process by which a surgeon creates a milieu that will ultimately lead to development of a bony bridge between to separate vertebrae, uniting them into a unit that physiologically behaves as a single bone. The indications for the creation of such a fusion are multiple, and include trauma, tumor, degenerative processes, spondylolisthesis, and other pathologies. The common denominator in all of these pathologies is spinal instability; some of these conditions, such as trauma, tumor, and spondylolisthesis result in frank spinal instability. In other situations, such as advanced degenerative disease or recurrent disc herniation, surgery has been performed that likely will result in spinal instability, and fusion can be justified as a prophylactic measure.

A universally-accepted definition of spinal instability has yet to reach consensus, but most authors agree that this condition involves some element of “excessive” anteroposterior or lateral movement of one or more vertebrae with respect to the remainder of the spinal column, and in particular, to the vertebrae immediately adjacent to the diseased or injured segment. The difficulty in establishing a uniformly-accepted definition is the arbitrary nature of the term “excessive.” The first problem that presents itself when attempting to define “excessive” is the fact that it has been, without question, established that there is an element of movement that exists between each of the vertebrae in the cervical, thoracic and lumbar spine as well as at the occipito-cervical and lumbosacral junctions. Defining the parameters of this acceptable, natural, and in effect necessary movement begets the controversy. Most authors would clearly agree that the evolution of injury to the spinal cord or nerves unambiguously constitutes “excessive “movement, with associated spinal instability. Radiographic demonstration of spinal instability, as defined by a set of generally-accepted parameters, is another unambiguous example of spinal instability. However, other signs, symptoms, physical and radiological findings that may suggest an element of spinal instability remain controversial and certainly, the concept of “prophylactic” fusion in association with decompressive surgery is argued by many experts.

Once a patient is found to have evidence of spinal instability, several therapeutic options exist. However, the most widely-recognized surgical procedure to treat this malady is fusion at the level[s] of the unstable segments. This may be undertaken from either an anterior or posterior approach. Posterior approaches are, in general, more common. The most common posterior approach that is utilized is the intra-transverse procedure, also known as the posterolateral fusion procedure, first described by Watkins in 1953. This is accomplished in part by placing the fusion material, or bone graft, in the space between the two transverse processes. The fusion material or bone graft, can either be osteo-inductive or osteo-conductive. Osteo-inductive material causes or “induces” bone growth and refers to material such as autologous bone which is frequently obtained from the patient's hip, as well as bioactive materials such as bone morphogenic protein [BMP], which can cause bone growth into a tissue bed into which the material has been laid.

Osteo-conductive materials include the use of material such as cadaveric bone, or even hydroxyapatite. Such substances do not, in and of themselves, induce bone growth, but will act as a scaffolding into which new bone can grow from exposed areas of cancellous bone such as denuded transverse processes.

In terms of obtaining bone graft material, a number of options are now available. Many surgeons still believe that the best source of such graft material is the patient's hip and iliac crest. Another option is to utilize cadaver bone to enhance and promote the fusion; although this is widely used, it remains somewhat controversial. Additional options continue to be developed.

It is also to be remembered that spinal fusion procedures are commonly performed in conjunction with procedures that remove part or all of the posterior bony elements of the spine. This component of the surgical procedure is referred to as decompression.

One option that, for unclear reasons, is not widely utilized is the use of the bone obtained during the decompressive component of the procedure. This is referred to as “local” bone. In particular, the spinous processes can serve as an excellent source of autologous bone. In fact, several recent studies have demonstrated that at least for single level fusion (i.e., L4-5 fusion) this source of bone is equivalent to hip bone in terms of the quality and maturation level of the fusion that forms. In order for the spinous process to be used, the process is first removed en masse by a large osteotomy instrument, or bone-cutting forceps. The isolated spinous process must then be divided into multiple smaller fragments. At present, no such instrument or device exists which serves the dual purpose of removal and division of the spinous process simultaneously. Such a device could also be utilized at other sites in which bone graft is obtained, such as the anterior/superior iliac spine (more often referred to in common parlance as the “iliac crest.” Therefore, the need exists for such a device. This concept is unique, useful, novel, and non-obvious.

### SUMMARY OF THE INVENTION

The invention is, therefore, provided bearing in mind these issues, needs, and considerations, and, as such, the objects of this invention can be achieved by providing a device in which multiple osteotomy blades are contained within a cartridge that can be, in turn, loaded into a deployment device. In the primary embodiment, it is proposed that the device is provided with a leading end, a central portion, and a trailing end, and incorporates a single or multiple-action leverage system. The leading end is represented by a set of at least two arms, the long axis of which would be parallel to the long axis of the device. Moreover, these arms are designed to be placed parallel and on either side of the bone to undergo osteotomy; in other words, the device, will ideally straddle the target bone, such as the spinous process, with the arms of the leading end. Furthermore, at least one of these arms is designed, in the preferred embodiment to be provided with a frame which shall house a cartridge. A series of osteotomy blades are then provided within this cartridge. The osteotomy blades are configured to create either horizontal or vertical osteotomies, or a combination thereof. Furthermore, the thickness of the bone fragments resulting from these osteotomies may also vary, at the discretion of the surgeon. The thickness is determined by the pre-set intervals of the grid of osteotomy blades that are secured into the cartridge. Additionally, the configuration of the grid will determine the shape of the bone fragments that result from the deployment of the device. The configuration of the bone fragments can, therefore, may vary from horizontal slices to squares/oblong components.

In the preferred embodiment, the frame disclosed above is found on the leading end of one of the arms. The leading end of the other arm is provided with a flattened surface, such that when these two arms are compressed together, the surface acts as a countermeasure against which the frame/cartridge complex creating the multiple osteotomies can be brought. This, furthermore, provides a counter surface, thus allowing the action of the osteotomy blades to be realized. The frame may be square, rectangular, ovoid, or of any geometric configuration. Ideally, the flattened surface would be of a complimentary configuration.

Variations and alternative embodiments can be contemplated by those skilled in the art. Such alternative embodiments would include the frame housing the cartridge on both sides as well as another embodiment in which the leading most aspect of one of the arms is provided with the frame while the trailing aspect of that is provided with a flattened surface, with a complimentary configuration being provided to the other leading arm. All such embodiments are included within the spirit and scope of this application.

The cartridge, as well as the frame on the leading end of the handle may or may not be reversibly secured to the leading end. In one, preferred embodiment, the frame is monolithic with the leading end of the device. The general configuration of such a frame typifies the general configuration of the frame throughout the various embodiments. This is an essentially square or rectangular-shaped structure in which the trailing end, which is confluent with the arm of the leading end of the device, is a closed, flat structure. The perimeter of this structure is then confluent with perpendicular sides. The size of these sides determines the size of the bone fragments that shall ultimately be harvested. The leading end of the frame is open such that it can interface with the bone to be harvested. The leading edges of the sides of the frame may or may not be sharpened edges that can contribute to the osteotomies. It is contemplated that ideally, the flat countermeasure shall also be monolithic with the other leading arm. It is expected that the cartridge containing the desired array of osteotomy blades is then inserted into the frame and the device is utilized.

An alternative embodiment is the instance wherein the frame is reversibly attached to the leading end of the handle, the frame may contain, as part of its embodiment, the grid composed of osteotomy blades. Alternatively, this grid may be secured within the frame prior to securing the frame to the leading end of the handle. If the frame is irreversibly attached to the leading end of the handle, then, clearly, the grid must be secured into the handle prior to deployment. Any combination of such an arrangement is also within the spirit and scope of this invention.

In yet another embodiment of the frame, the frame is composed of two elements which couple and uncouple with the deployment of the device. As such, in this embodiment, the sides of the frame containing the grid will separate from the base of the frame during deployment and will reunite the base when the device is returned to the primary, uncoupled position. In this embodiment, it is envisioned that there are protrusions arising from the base of the frame. These protrusions are positioned such that when the device is deployed, obtaining bone fragments within the frame, and then returned to the non-deployed positioned, these projections will “eject” the bone fragments obtained from the frame.

Furthermore, in an alternative embodiment a frame is found on both sides of the leading end of the device. This, again, can be reversibly or irreversibly secured to the leading end of the handle.

The central portion of the device serves as the junction which communicates the leading end of the device with the trailing end of the device. As has already been disclosed, the leading end of the device serves to accomplish the multiple osteotomies as the result of deployment of the device. The trailing end of the device, as to be disclosed below, serves as the deployment mechanism. The central portion of the device serves to translate the deployment activated by the trailing end to the leading end, which then, in turn, accomplishes the multiple osteotomies.

The central portion of the device is composed of a single or multiple action or leverage system by which the deployment achieved at the trailing end of the device is translated to the leading end of the device, thus permitting the device to accomplish the osteotomies. In the instance where a single leverage mechanism is utilized, it is noted that the device itself, when viewed from the frontal plane, actually represents a modified “X-shape” device. As such, it is noted that in this embodiment, the device is represented by two monolithic components that intersect at the mid section. Each component has a leading end, the leading arms which have been disclosed previously, this leading end then being somewhat curved in such a fashion that the element approaches the other element and crosses it at a pivot point, and then continues towards the trailing end, again oriented in the same general direction as the leading end of the device. The device can also be viewed as two monolithic elements that intersect at the mid position, with arms extending substantially perpendicular from the intersection point. It can be seen, therefore, that when the trailing end is deployed by drawing two arms or handles that are found on the trailing end towards each other, the intersection will provide leverage as the leading ends are drawn towards each other, thus accomplishing the osteotomies.

It can be appreciated that achieving an osteotomy is not ergonomically optimal, and therefore, this can be resolved by providing the central portion of the device with the so-called “double-action” mechanism. Such a mechanism, frequently found in such instrumentation and devices provides for a series of intersecting arms in the mid section. These arms are arranged in an ergonomic and mechanical fashion such that when the handles of the trailing end are drawn towards each other, these arms will interface in such a way as to draw the arms of the leading end of the device towards each other, with substantially increased leverage. This is specifically created by an arrangement in which a counterbalance spring is positioned between the two deployable handles on the trailing end of the device. In this mechanism, the device itself is composed essentially of four monolithic elements. The two deployable handles on the trialing end of the device each represent one of these monolithic elements, with the two arms of the. leading end of the device representing the other two monolithic elements. There is a pivot point at the leading end of the two deployable handles causing the leading-most ends of these handles to be drawn apart when the trailing end of the handles are compressed towards each other. Leverage is added to this compression by the counterbalance spring positioned between the two handles. At the junction of the monolithic components comprising the handles with the monolithic components comprising the arms of the leading end, another pair of pivot points are located. Yet another pivot point is positioned between the two arms of the leading end which are configured such that in the primary non-deployed position, the two arms of the leading end are distracted from each other. When the deployable handles on the trailing end of the device are drawn towards each other, the arrangement of these pivots cause the leading ends of these elements to be drawn or compelled away from each other. Because of the pivot point between the two arms of the leading end, in combination with the configuration of the arms of the leading end, this action causes the leading-most aspect of the device to be compelled forcefully towards each other. In the non-deployed position, these jaws are actually distracted from each other, and as the deployable handles on the trailing end approximate each other, through the mechanism thus described, the components of the leading end will then approximate each other. This is typified in medical devices and instruments such as a Lexell or a Hoarsely bone cutter, but other examples of this can be found in both medical and non-medical industry and the use of such a mechanism has been widely established. A multiple action center point which would generate additional leverage can also be contemplated and is also included within the spirit and scope of this invention.

As has been alluded to in the previous paragraphs, also herein disclosed is a trailing end of the device which is, in the preferred embodiment, composed of two handles that can be manually operated by the surgeon. According to the leverage mechanism that has been provided herein, these handles can be drawn towards each other, or compelled away from each other in terms of activating a device.

Regardless of the embodiment, once the grid has been secured into the frame on the leading end of the handle, the device is ready for deployment. The device is brought into a position and parallel to the long axis of the target bone. If the target bone is the iliac crest, for example, it would be expected for a sufficient amount of tissue attachment on both sides of the proposed site of harvest are cleared, and the device is then brought in the segment of anterosuperior iliac spine and the device is deployed. In the instance wherein the spinous process is the target bone, the device is brought in perpendicular to the long axis of the spine, again parallel and straddling the long axis of the spinous process. Regardless of the embodiment by which the handles are deployed by the surgeon, the resultant effect causes causing the arms of the leading end of the handle to approximate one another. With this action, the arms on the leading end will close, and the osteotomy blades accomplish both removal of the spinous process and creation of multiple bone fragments which are then available for use as graft material. The inventors contemplate the use of the device in other sites whereupon bone graft may be harvested.

Another alternative embodiment is also provided. In this embodiment, a pneumatic drive is utilized rather than a manual drive. This embodiment requires some modification of the central portion and trailing ends of the invention. The leading end will be substantially similar to the leading end that was disclosed in the manually driven device. That is to say that the leading end of this embodiment also is provided with two arms which are designed to straddle the target osseous structures such as the spinous process with the anterior/superior iliac spine. Furthermore, these arms are again provided with monolithic or detachable frame into which a permanent or detachable cartridge is found. This cartridge, furthermore, houses multiple osteotomy blades used to create multiple bone fragments.

In the pneumatically driven device, there may be one or more handles found on the trailing end, but there would not be a requirement for two handles which would be either brought together or compelled apart as disclosed in the manually deployed embodiment above. In the preferred embodiment of the pneumatically driven device, a single handle is found. This handle, furthermore, is provided with a port into which a standard pneumatic tubing system can be connected, allowing for compressed air typically available in the operating room to be passed to the device. The port at which the compressed air enters would be found at the trailing-most end of the trailing end, or it may be found attaching to a portal at any point along the shaft of the trailing end. The trailing end of the device then leads into the pneumatic mechanism housing center. This provides for intake of the pressurized air and converts the energy from the pressurized air to the mechanism that drives the approximation of the two arms on the leading end towards each other, thus accomplishing the goal of removal of the bone and achievement of multiple osteotomies. Within the air intake drive mechanism is found a chamber into which the pressurized air enters. That chamber is oriented substantially perpendicular to the long axis of the arms of the leading end of the device. Also found within the air entry chamber are two pistons which are again oriented perpendicular to the long axis of the arms of the leading end. Furthermore, these pistons are coupled with, or are monolithic with the trailing end of these arms. The arms then extend out from the air intake drive mechanism housing unit, the arms extending in their course towards the leading end of the device. At a point along the shaft of the arms of the leading end, a pivot pin is provided. The pivot pin is substantially perpendicular to the long axis of the device, and is connected to the leading end of the pneumatic mechanism housing center. The pivot pin is positioned such that the arms of the leading end rotate about the pivot pin in a fashion such that the leading-most ends of these arms will rotate towards or away from each other. The pistons within the pneumatic mechanism housing chamber will also move towards or away from each other in a reciprocal fashion to the movement of the leading-most ends of the arms. Hence, as such, in the primary, non-deployed position, these pistons are approximating each other within the air intake chamber. As the air under pressure is brought into the chamber, these pistons are driven away from each other. Owing to the reciprocal relationship between the position of the pistons and the position of the ends of the leading arm, this would thus drive the ends of the leading arm towards each other. Hence, if said frame and cartridge are connected to one leading end, with the flattened countermeasure present on the other leading end, as these leading ends are driven towards each other, the desired osteotomies will be achieved.

In an alternative embodiment, the trailing ends of the arms of the leading end of the device are again found within the drive mechanism housing element. In this embodiment, a manifold will equally distribute the pressurized air as it enters the mechanism. the air, which is under high pressure is distributed over the lateral aspects of the trailing ends of the arms. This drives the trailing end of each arm towards each other. As there is no pivot pin or rotating mechanism, as was described in the previous embodiment, this would in reality drive both arms towards each other and result in accomplishing desired osteotomies.

Other embodiments of the pneumatic-type mechanism can be contemplated by those skilled in the art. Such embodiments are also incorporated and included with the spirit and scope of this application.

Other alternative embodiments can also be contemplated, including other sources of power-assisted mechanisms. These could include an electronic drive mechanism as well as a hydraulically-driven mechanism.

While the invention has been shown and described with reference to certain preferred embodiments, it will be understood by those skilled in the arts that various changes and modifications in form and detail may be made therein without departing from the spirit and scope of the invention as defined herein.

### BRIEF DESCRIPTION OF THE DRAWINGS

FIG. 1A—Lateral view of a single-action embodiment of the device; the device is fully deployed in this view

FIG. 1B—Top view of single-action embodiment of the device

FIG. 2—Lateral view of a single-action embodiment of the device non-deployed

FIG. 3—Lateral view of a double-action embodiment of the device in the non-deployed state

FIG. 4—Lateral view of a double-action embodiment of the device with the device fully deployed

FIG. 5A—Lateral view of frame on leading end of the device

FIG. 5B—Top view of frame with grid of osteotomy blades in place

FIG. 5C—Elevational view of frame with osteotomy blades in place

FIG. 6—Exploded view of frame with cartridge of osteotomy blades

FIG. 7A—Lateral view of countermeasure component of leading end of device

FIG. 7B—Top view of countermeasure component of leading end of device

FIG. 7C—Elevational view of countermeasures component of leading end of device

FIG. 8A—Transaxial view of typical lumbar vertebra with device being positioned to straddle the spinous process

FIG. 8B—Device deployed and spinous process removed, and fragmented within frame of device

FIG. 8C—Bone fragments being emptied from cartridge

### DETAILED DESCRIPTION OF THE DRAWINGS

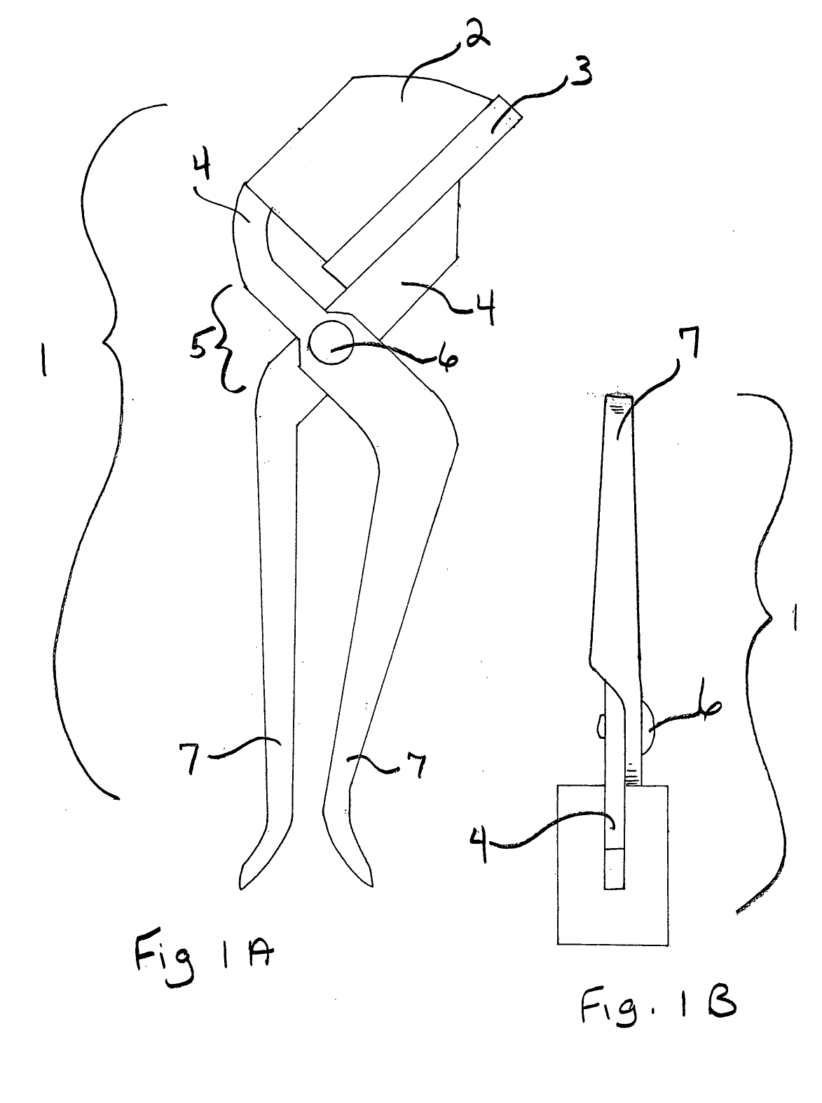

Referring now to the drawings in which like reference numerals identify similar or identical elements throughout the several views, and in particular, to FIG. 1A, where upon a lateral view of a single-action embodiment 1 of the invention is depicted. In this view, the leading arms of the device 4 have been approximated in a position consistent with a fully deployed position of the device 1. Also seen in this view is a lateral view of the frame 2 which is found on one of the two arms 4 of the leading end of the device. A countermeasure 3, against which the frame 2 has been brought, is found on the other leading arm 4. The leading arm 4 on which the frame 2 is coupled has been curved so that the device 1 can be deployed by approximating the deployable handles 7 on the trailing end of the device 1. In other embodiments (not shown) the leading ends 4 are approximated by distracting the deployable handles 7. In this embodiment, the single action mechanism 5 governed by the pivot point 6 will lead to approximation of the leading arms 4 with approximation of the deployable handles 7.

FIG. 1B demonstrates a top view of the same device 1. In this view, a top view of the frame 2, leading arm 4 and pivot point 6 can be seen. Also seen is a top view of the deployable handle 7.

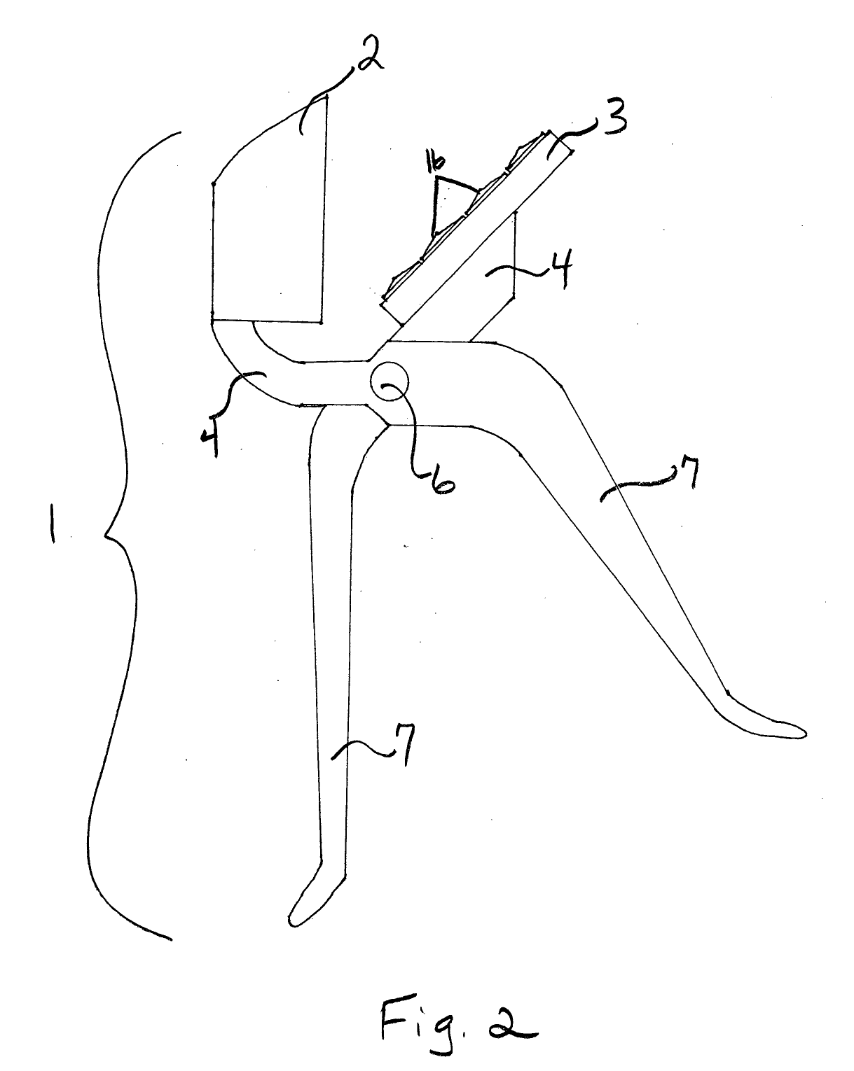

FIG. 2 demonstrates a lateral view of a single-action embodiment of the device 1 in the non-deployed position. In this view, the deployable handles 7 have been distracted away from each other. As the result of the pivot point 6, the leading arms 4 are also distracted away from each other. Again seen is the lateral view of the frame 2, as well as the lateral view of the countermeasure 3. Also seen is the corrugated surface 16 of the countermeasure 3. The device is now in proper position to be inserted around a target osseous structure such as a spinous process.

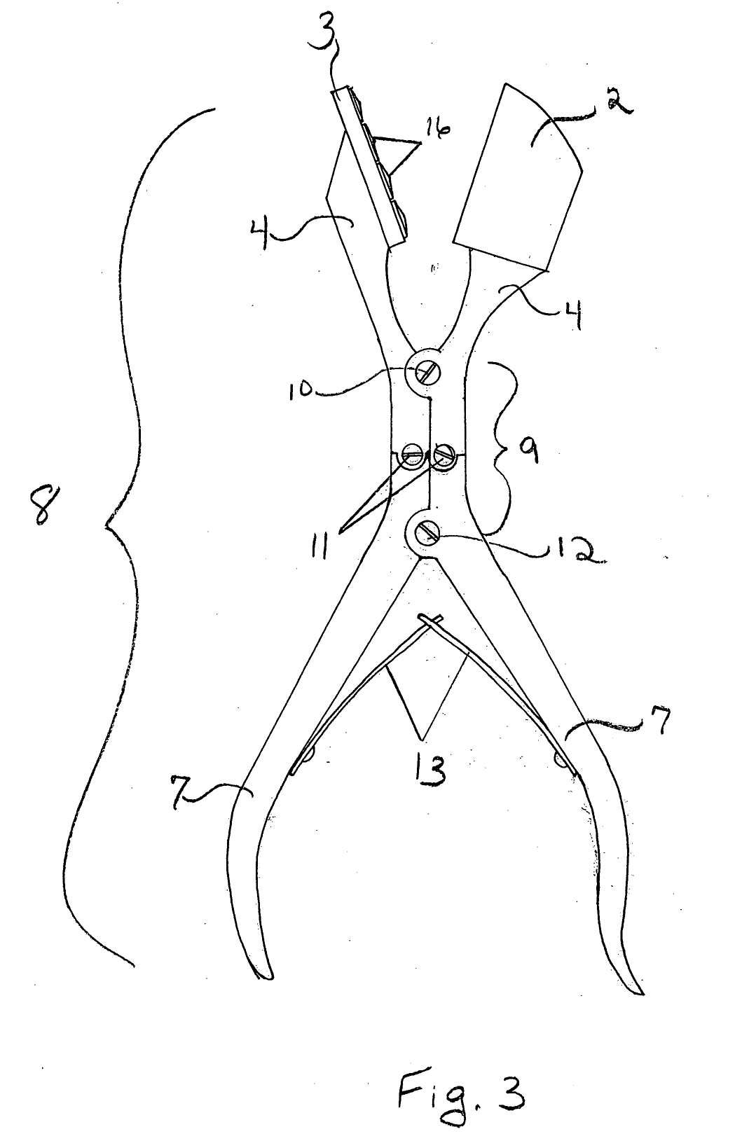

In FIG. 3, a lateral view of a double-action device 8 is depicted. In this embodiment, the frame 2, as well as the countermeasure 3 with its corrugated surface 16 are seen. Also seen are the arms of the leading end 4 and the deployable arms of the trailing end 7. The double-action mechanism 9 is composed of a leading pivot 10, two intermediate pivots 11, and a trailing pivot 12. A counterbalance spring 13 creates tension between the deployable handles 7 as they are drawn towards each other, thus adding leverage to the double-action mechanism 9.

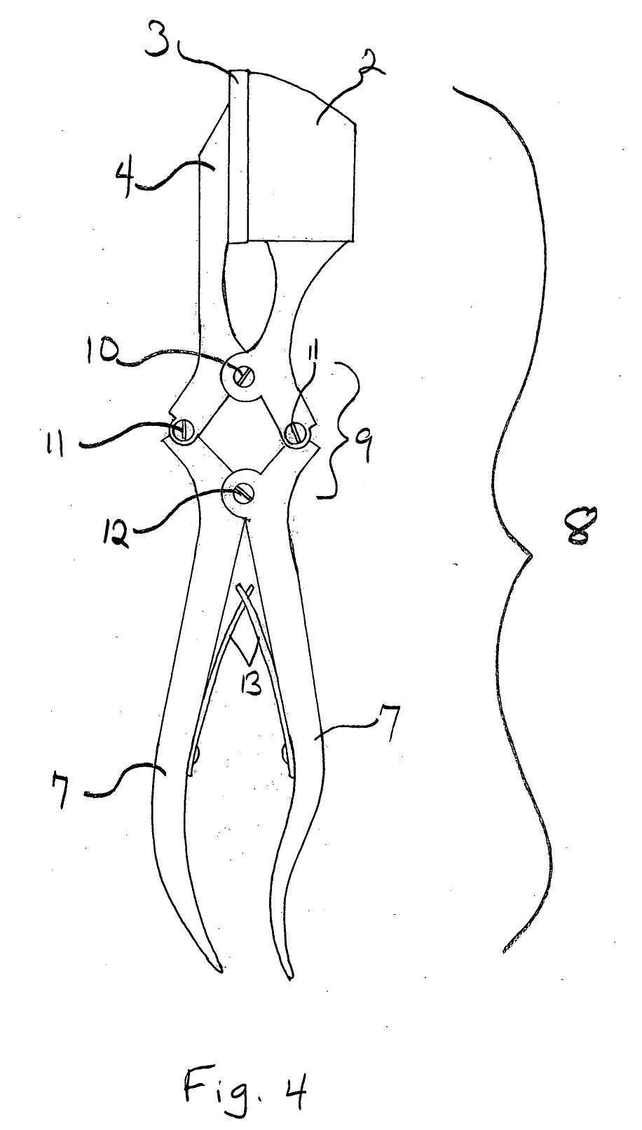

As seen in FIG. 4, when the deployable handles 7 of the double-action device 8 are drawn towards each other, the actions of the counterbalance spring 13, in concert with the trailing pivot 12, the intermediate pivots 11, and the leading pivot 10 all result in approximation of the leading arms 4. This causes the frame 2, with its cartridge of osteotomy blades (not shown) to be forcefully compelled against the countermeasure 3. This results in removal of the target osseous structure as well as simultaneous fragmentation of this structure into bone fragments, the size of which are dictated by the grid of osteotomy blades.

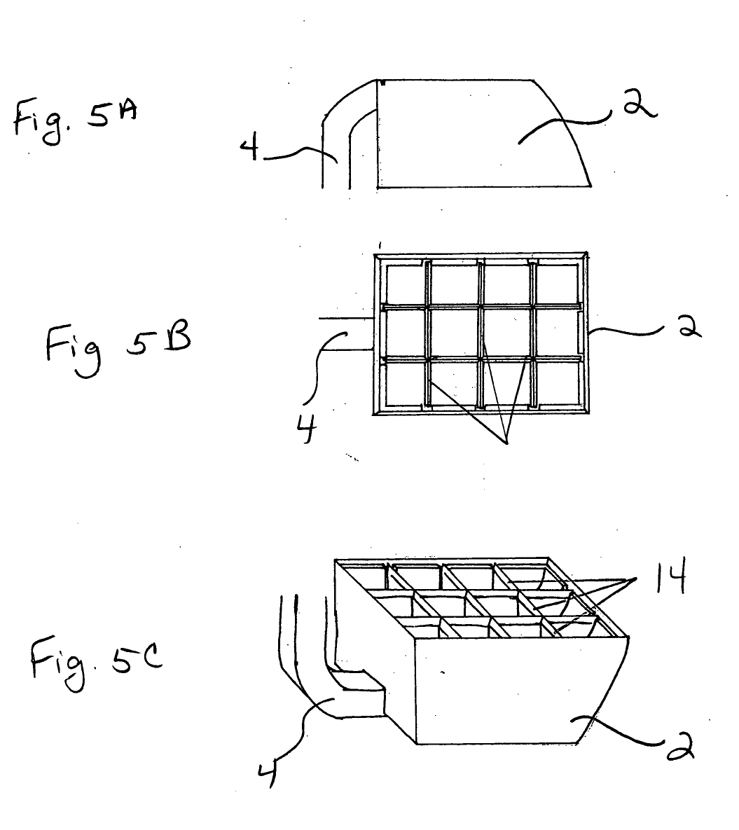

In FIGS. 5A, 5B and 5C, the frame 2 along with the osteotomy blades 14 are illustrated. FIG. 5A demonstrates an arm of the leading end 4 which has been coupled frame 2 as shown in the lateral view. FIG. 5B demonstrates a top view of the frame 2 with the cartridge of osteotomy blades 14 having been coupled within the frame 2. The leading arm 4 is again seen. In FIG. 5C, an elevated perspective view demonstrates the arm of the leading end 4 coupled with the frame 2, with the grid of osteotomy blades 14 having then been coupled within the frame 2.

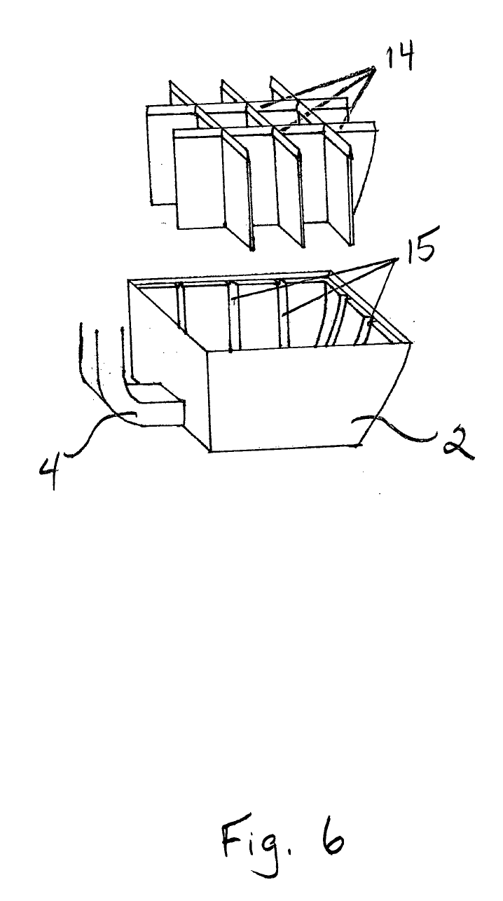

FIG. 6 demonstrates an exploded view of the frame 2 with the osteotomy blades 14. The grid of osteotomy blades 14 has been removed from the frame 2 and the interior of the frame 2 demonstrates slots 15 which may be provided to house the cartridge of osteotomy blades 14.

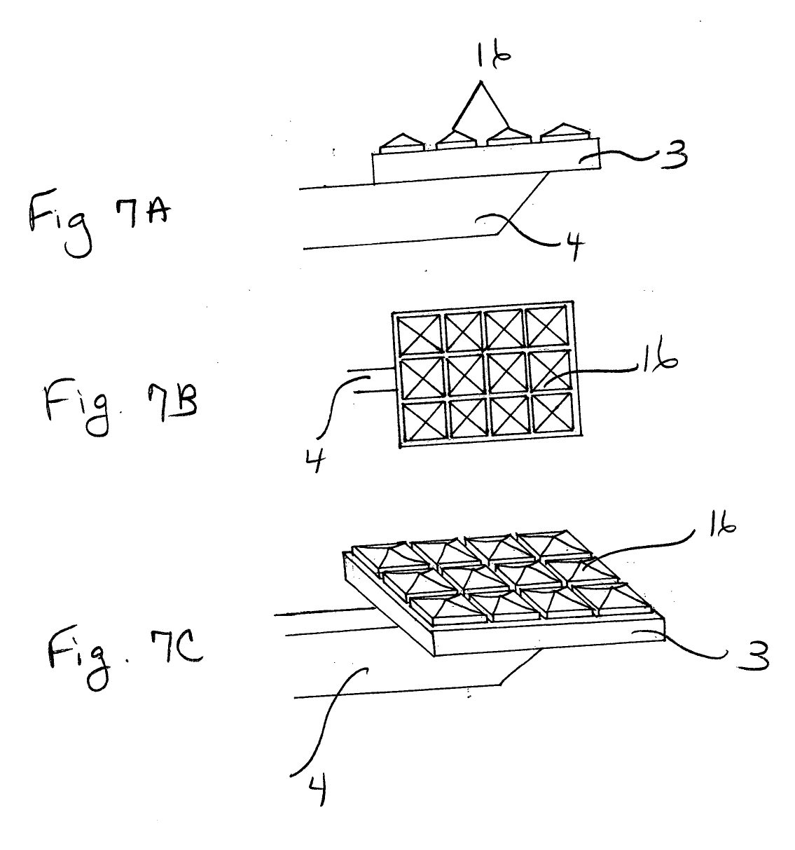

In FIG. 7A, a lateral view of the arm of the leading end 4 with the countermeasure 3 is seen. Also seen is the corrugated surface 16 of the countermeasure 3. FIG. 7B demonstrates a top view, again showing the arm of the leading end 4, as well as the corrugated surface 16. FIG. 7C demonstrates an elevated perspective view of the same structures, showing the arm of the leading end 4 with the countermeasure 3 and the corrugated surface 16.

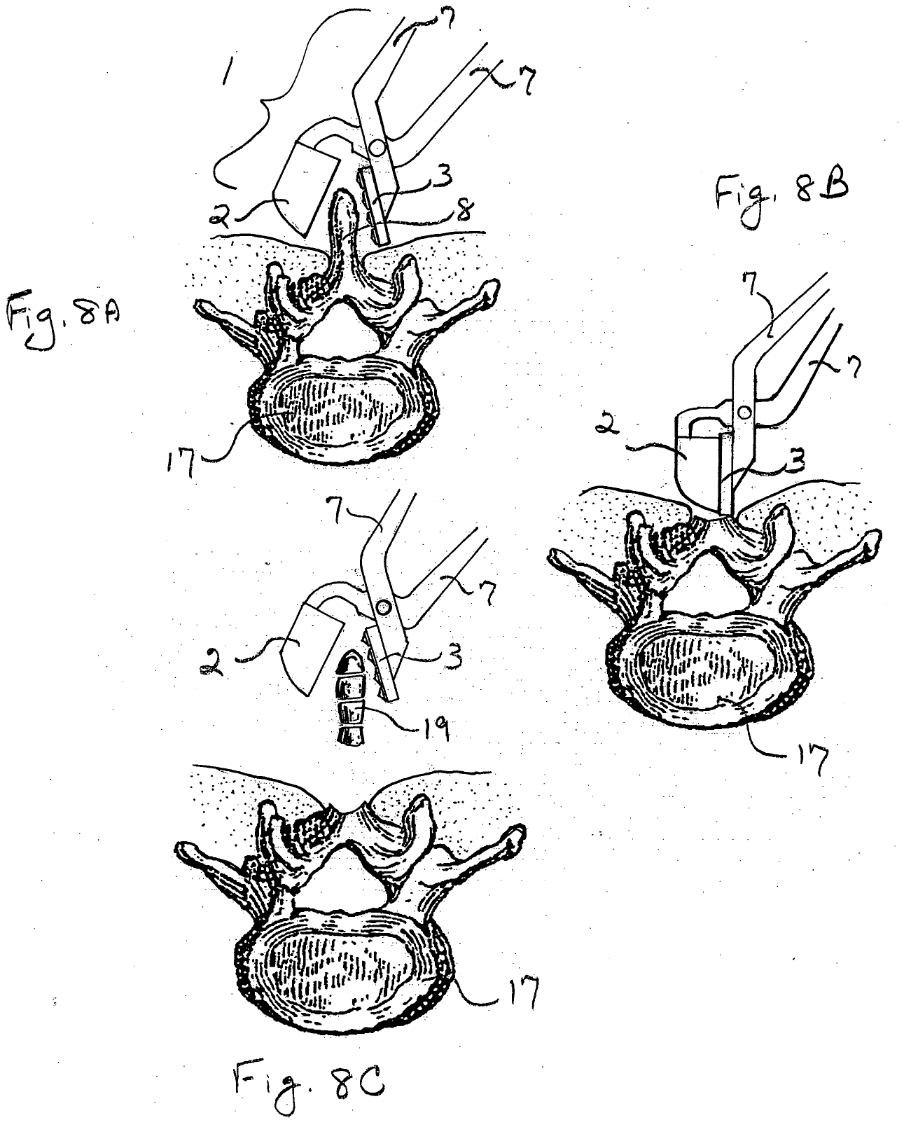

FIGS. 8A, B and C, demonstrate a single-action embodiment of the device 1 as it would be utilized to resect and fragment a spinous process 18. In FIG. 8A, a transaxial view of a vertebra 17 with its respective spinous process 18 is seen. The device 1 is being positioned with the handles 7 in the non-deployed position. This permits the frame 2 and countermeasure 3 to straddle the spinous process 18. In FIG. 8B, the device 1 has been deployed and as such the frame 2 has been drawn against the countermeasure 3. This has resulted in removal or resection of the spinous process 18. In FIG. 8C, the device 1 has been returned to the non-deployed position. With the distraction of the frame 2 from the countermeasure 3, the fragments 19 of the spinous process 18 are removed from the device 1.

What is claimed:

1. A system of devices, used during surgery, that results in the removal of a portion of a target bone with the simultaneous fragmentation of that portion of the target bone of the patient being operated upon, or a donor, consisting of:
A leading end, a central portion, and a trailing end;
The leading end of the device shall have a system of at least two arms, at least one of which is provided with a frame in which a cartridge containing a series of osteotomy blades have been positioned, these blades oriented to produce bone fragments as the target bone is removed;
A central portion which houses the deployment/actionator mechanism;
The trailing end of the device, which shall be composed of a pair of deployable handles;
The deployable handles of the trailing end may be deployed be either a single-action or a double-action mechanism, using either manual, mechanical, pneumatic, electrical, or any other means of providing adequate power form their deployment.

2. The primary embodiment of the leading end of the device in claim 1, consisting of:
At least two arms that are oriented parallel to each other with their long axis oriented in continuation with the long axis of the device;
The at least two arms are oriented so that they may straddle a segment of target bone donor site;
At least one of the arms is provided with a frame which is square, rectangular, oblong, ovoid, or of any geometric shape;
A flattened surface or countermeasure is provided on the leading end of the arm apposing the arm that has been provided with the frame.

3. The frame in claims 1 and 2, which is configured in such a fashion that a cartridge may be reversibly or irreversibly secured within this frame.

4. The cartridge in claims 1 and 3 that can be reversibly or irreversibly secured within the frame in claims 1 and 2, which is composed of a series of osteotomy blades, these blades arranged in such a fashion so that they are parallel to the long axis of the device, as well as perpendicular to the long axis of the device and to each other.

5. The cartridge in claims 1, 3 and 4, which is composed of a series of osteotomy blades arranged in such a fashion such that when deployed against the target bone, these osteotomy blades will produce the desired bone fragment to be used for the bone graft.

6. The countermeasure in claim 2, which is found on at least one arm of the leading end in claims 1 and 2 and which serves as a base against which the osteotomy blades in claims 1, 3, 4 and 5 can be brought when the device is fully deployed; the surface of the countermeasure may be flattened or corrugated in a fashion that is complimentary to the grid of osteotomy blades.

7. The osteotomy blades in claims 2-5, which are composed of thin, flat structures of any geometric shape and which are provided with an edge of sufficient sharpness to create an osteotomy in bone on at least one side.

8. The trailing end of the device in claim 1 which is a deployment mechanism composed of at least two handles which can be manually deployed by either drawing these handles towards each other or away from each other, dependent on the leverage mechanism of the central portion of the device.

9. An alternative embodiment of the deployment mechanism of claim 8 consisting of a pneumatic drive utilized to compel the leading arms of the invention towards each other.

10. An alternative embodiment in which there is an intake port to receive pressurized air.

11. The pneumatic drive mechanism in claims 9 and 10 by which the interface between the trailing ends of the arms of the device and the compressed air results in approximation of the leading ends of the arms accomplishing the desired osteotomy.

12. The interface between the compressed air and the arms of the leading end in claim 11 which provides for a chamber in which the interface between the trailing ends of the arms and the compressed air occurs.

13. The interface between the air and the trailing ends of the leading arms in claims 10 and 11 by which the trailing ends of the arms are provided with pistons encased within the chamber in claim 12 oriented substantially perpendicular to the long axis of the arms.

14. The pistons in claim 13 which are found within the air intake chamber are oriented within the chamber in claim 12 such that these pistons will be driven apart with the introduction of the pressurized, compressed air.

15. The part of the pneumatic mechanism in claims 10-14, pistons are provided on the trailing end of the arms.

16. As part of the pneumatic mechanism in claims 10-15, there is a fulcrum pin provided along the shaft of the arms of the leading ends.

17. The fulcrum pin in claim 16 is arranged such that the long axis of the arms of the leading end are provided with a moment of rotation around the fulcrum pin in a fashion such that when the pistons are driven apart, the leading-most ends of the arms are driven towards each other.

18. An alternative embodiment of the pneumatic drive, consisting of:
A pressurized air intake valve, by which there is a manifold responsible for distributing the compressed air entering the mechanism;
The compressed air enters a chamber on the lateral aspect of the trailing end of each arm;
This ultimately results in the two arms being driven towards each other.

19. An alternative embodiment in which frames are found on both arms of the leading end such that both frames can accommodate cartridges as described above and when the device is fully deployed, the osteotomy blades in each of these cartridges will approach each other.

20. An alternative embodiment, in which there is an additional mechanism found on the arms of the leading end of the device bilaterally that extrudes the bone fragments from the cartridge once the arms of the leading end are disengaged from the deployed to the primary position.

21. An alternative embodiment, in which there is an auxiliary device that may be reversibly secured to the leading end of the primary device, this auxiliary device being fashioned so that when it is deployed, it will remove the bone fragments from the cartridge.

22. A freestanding device that assists in the removal of the bone fragments from the cartridge.

23. The central portion of the device in claim 1, which is composed of a single or multi-pivot system that translates the action of the deployable handles to the approximation of the arms of the leading end of the device.
